(19) United States
(12) Patent Application Publication
(10) Pub. No.: US 2005/0175721 A1
(43) Pub. Date: Aug. 11, 2005

(54) Method of augmenting the immune-modulatory activity of standardized Echinacea preparations

(76) Inventors: Ernesto A. Brovelli, Corona, CA (US); Haeri Roh-Schmidt, Ada, MI (US); Angela Brackhahn, Hood River, OR (US)

Correspondence Address: ALTICOR INC., 7575 FULTON STREET EAST MAILCODE 78-2G, ADA, MI, 49355, US

(21) Appl. No.: 10/774,092

(22) Filed: Feb. 6, 2004

(51) Int. Cl. A61K 35/78; C12Q 1/00

(52) U.S. Cl. 424/737; 435/4; 47/58.1 R

(57) ABSTRACT

Echinacea preparations having standardized chicoric acid levels and inducing an augmented level of immune-stimulatory activity. Augmentation of the immune stimulatory activity is achieved by the selection of a harvest time for Echinacea plants that is during a plant maturation stage that is prior to full bloom. Methods for augmenting the immune-stimulatory effects of Echinacea extracts by harvesting Echinacea plants prior to full bloom.

### BACKGROUND OF THE INVENTION

Extracts from plants of the Genus Echinacea have been used to modulate the immune response in mammals. Echinacea plants are perennial plants that produce a coneflower and are capable of yielding a crop for several years after an initial planting. Echinacea plants may require two years to become established. The plants of each new crop progress through several maturation stages prior to harvest. During stages 1 through 7, Echinacea plants progress from the vegetative stage through bud and flower development and then to full bloom. Echinacea crops are typically harvested at full bloom and are processed to extract products having medicinal properties.

The specific Echinacea compounds responsible for modulating an immune response in mammals are unknown. However, Echinacea plants contain numerous active phytochemicals, such as caffeic acid derivatives (e.g., chicoric acid), alkylamides (e.g., dodecatetraenoic acid isobutylamides), and glycoproteins/polysaccarides. It is advantageous to consume the full range of the phytochemicals in Echinacea in order to gain the beneficial effect of the combination. Elimination of one class of constituents could reduce the beneficial effect. Botanical extracts are standardized to contain specific levels of marker compounds. Typically, Echinacea extracts are standardized to contain a known concentration of one or more of chicoric acid, polysaccharides or alkylamides.

### SUMMARY OF THE INVENTION

One embodiment of the methods described herein is a method for determining optimal harvest window of medicinal plants by harvesting at least one plant from each of a number of different maturation stages for the plant; adding a preparation of the plant to a cell culture; harvesting the cell culture; analyzing the cell culture for a level of a product the medicinal plant induces; and observing the level of the product corresponding to each of the different maturation stages.

Another embodiment of the methods described herein is a method of augmenting the immune-modulating effects of Echinacea by harvesting the Echinacea plant during a maturation stage prior to full bloom. Preferably the Echinacea plant is harvested during or prior to stage 6 maturation. Stage 6 is characterized by erect ligular flowers that may be green or white in color. More preferably, the Echinacea plant is harvested during or prior to stage 3 maturation as characterized by the plant having a diminutive bud size of about 18 mm. Most preferably, the plant is harvested during the vegetative stage (maturation stage 1).

Still another embodiment of the invention described herein is an Echinacea preparation that has a standardized concentration of chicoric acid and an augmented level of immune-stimulatory activity wherein the preparation was obtained from an Echinacea plant harvested during a maturation stage prior to full bloom.

The term “augmentation” is used to refer to an increase in observed activity.

The term “bracts” refers to a modified leaf or leaflike plant part that protects the inflorescence.

The terms “immune-modulatory” and “immune-stimulatory” are used to refer to a change in the level of production of messenger ribonucleic acid (mRNA) transcripts or proteins associated with an immune response.

The terms “induce” or “induction” (e.g., induction of immune cytokine mRNA) refer to an increase of the total measurable transcription or translation product or activity as measured by quantifying RNA or protein levels.

The term “inflorescence” refers to a flowering part of a plant.

The terms “ligule” or “ligular flower” refer to the corolla of a ray floret of a composite flower.

The terms “maturation stage” or “stage” refer to a step in the development process of plants during each annual cycle. These terms do not refer to age (in years) of a perennial crop.

The term “meristem” refers to tissue from which new cells are formed.

The term “preparation” refers to Echinacea plant products that result from the dehydrating and/or powdering of plant material or from the chemical extraction of plant material.

### BRIEF DESCRIPTION OF THE DRAWINGS

FIG. 1 is a bar graph depicting the levels of mRNA for cytokines IL-1a, IL-1b, IL-6, IL-8 and IL-10 and levels of IFN-g, MIP-1 and TNF-α produced by THP-1 cells after treatment with extracts from Echinacea plants harvested at maturation stages 1, 3 and 6 as compared to a standard lipopolysaccharide (LPS) solution used as a positive control.

### DETAILED DESCRIPTION OF THE INVENTION

Uses of medicinal plants, such as Echinacea, are typically based on, or derived from, the use by the original discoverers of those plants. The original users of Echinacea selected particular plants for use based on empirical information. For example, early users of Echinacea likely knew when to harvest a plant based on visual cues as to a plant's readiness to provide a desired result. Recently, a more sophisticated approach has evolved. This approach consists of conducting chemical analyses for the presence and level of one or more marker compounds.

Chicoric acid is a preferred marker compound for Echinacea purpurea preparations because this marker is valuable for its use in species identification. However, no definitive connection exists between any specific marker alone and observed immune-modulating activity. Neither the empirical or chemical methods reveal optimal harvest window to achieve the most advantageous immune-stimulatory activity at the cellular level. Echinacea is typically harvested at full bloom. However, harvesting Echinaceacrops at earlier stages of plant maturation can provide better immune-modulation, or immune-stimulation, as given by gene response in human cells while maintaining good levels of marker compounds for standardization. The methods described herein provide for the determination of ideal harvest window to yield the optimal level of immune-stimulation by use of in-vitro assays to measure the induction of immune-stimulatory nucleic acids and/or proteins.

Echinacea purpurea plants progress through several maturation stages identified herein as maturation stages 1-7. Stage 1 is the vegetative stage. Stage 2 is characterized by hidden bud formation. In order to observe stage 2, the leaves surrounding the apical meristem must be unfolded to confirm the presence of bracts signifying the onset of inflorescence. Stage 3 is the diminutive bud stage during which the bud is approximately the diameter of a United States dime (18 mm). Stage 4 is the enlarged bud phase during which the buds are approximately the diameter of a United States nickel (21 mm). Stage 5 is characterized by the formation of a cone. The cone is a composite of densely arranged florets. In stage 6, ligules emerge and stand erect. The erect ligules of stage 6 form along the perimeter of the cone and are green or white. Stage 7, which is full bloom, is complete once the ligules elongate, droop downward and come into color. The color may range from pink to deep lavender.

At each maturation stage described above, whole E. purpurea plants were pruned at approximately 5 centimeters above the ground. Five centimeters above the ground is approximately the location at which Echinacea plants are harvested by machine in the field. The crop from which the plants were taken was six years old. Re-establishment of the plant occurs during the spring season. Stage 1 plants were harvested approximately 71 days after re-establishment of the plant. Stage 3 plants were harvested approximately 76 days post re-establishment. Stage 6 plants were harvested approximately 83 days post re-establishment. All aerial parts of the plant above 5 cm were collected, chopped into pieces of about 1-2 inches with pruners, dehydrated in a dryer at 54 degrees Celsius, ° C., (130 degrees Fahrenheit, ° F.) for 24 hours. The chopped and dried plants were then ground into powder with a coffee grinder. The particle size of the plant material may be reduced by a number of methods including chopping, shredding, grinding, milling or by other means.

Due to the natural variations in developmental pace among individuals, each individual plant developmental stage may vary from that of the adjacent plants. Ideally, 100 percent of the plants in a crop would be at the developmental stage desired for a harvest. However, due to the natural variations in developmental pace, about 80% of the crop plants may be at a particular stage at any one time. Stage 1 maturation has been observed to be about 90-100% uniform. Stage 3 is about 80% uniform and stage 6 is about 70% uniform. Current general practices are that Echinacea is harvested at full bloom. Harvesting at full bloom may cause collection of some plants that have begun the degradation process.

### HPLC Analysis

Dried plant material that had been dried and reduced in size was added to a solution of 80% methanol in water and heated to 60° C. (140° F.) for 1 hour while shaken. Subsequently, the plant material was place on a shaker at room temperature for 1 hour and then filtered and diluted as necessary for analysis.

Chicoric acid levels for plant extracts at each stage of maturation were determined by High Pressure Liquid Chromatography (HPLC). The HPLC system is equipped with a Photo Diode Array detector. The column used was Symmetry C18, PIN WAT054215, 4.6 mm×250 mm, Sum (micrometer) particle diameter by Waters Corp. (Milford, Mass.). The following conditions were used:

Instrument Parameters:

Mobile Phase:
0.2% phosphoric acid in water
100% methanol
100% acetonitrile

Solvent Program:

Gradient:

| Time | Solvent A | Solvent B | Solvent C |
|---|---|---|---|
| 0 | 70% | 20% | 10% |
| 25 | 54% | 36% | 10% |
| 30 | 25% | 40% | 35% |
| 35 | 70% | 20% | 10% |
| 45 | 70% | 20% | 10% |

Flow Rate: 0.6 mL/min.

Injection Volume: 10 uL

Column Temperature: room temp. 25° C. (77° F.); detection wavelength: 330 nm.

The results of the analysis for each maturation stage tested are shown in Table 1.

TABLE 1
Chicoric Acid Concentrations

Concentration of
Chichoric Acid (%)

| Stage | Mean | Standard Error |
|---|---|---|
| 1 | 3.49 | 0.09 |
| Vegetative | | |
| 2 | 3.52 | 0.16 |
| Hidden Bud | | |
| 3 | 3.26 | 0.10 |
| Diminutive Bud | | |
| 4 | 3.62 | 0.11 |
| Enlarged Bud | | |
| 5 | 3.36 | 0.12 |
| Cone Formation | | |
| 6 | 3.54 | 0.14 |
| Ligules Erect | | |
| 7 | 2.59 | 0.10 |
| Ligules with Color | | |

The levels of chicoric acid measured during maturation stages 1-6 for Echinacea purpurea are substantially similar or substantially equivalent. Echinacea plants harvested during maturation stage 7 are less commercially desirable due to the drop in the levels of chicoric acid observed at this stage. In other words, the variation between the chicoric acid levels listed in Table 1 are within the levels generally accepted by those skilled in the art to be variation between individual plants or to be variations within acceptable tolerances for these types of analyses.

As a plant-based medicinal product, standardization to a marker such as chicoric acid is important to meet market or regulatory expectations. Functionality of the Echinacea extracts as measured by gene induction varies with stage of maturation while marker level is essentially unaffected by variation in harvest time for stages 1-6.

### Gene Induction Assay

Plant material that had been dried and reduced to smaller-sized pieces by cutting and/or grounding was dissolved in a dimethyl sulfoxide (DMSO):ethanol:water mixture of 50:30:20 by volume initially. Subsequently, the extracts were further diluted with a desired amount of cell culture medium to obtain a particular concentration for use in the gene induction assay.

Three maturation stages of Echinacea purpurea were compared for their immune stimulatory effects on THP-1 monocyte/macrophage cell line. The THP-1 cell line is derived from human peripheral blood and can be obtained from ATCC, Manassas, Va. (ATCC No.: TIB-202). THP-1 monocyte/marcrophage cell line is used as a model for the circulating blood monocytes and macrophages in humans and other mammals. Circulating blood monocytes and marcophages play a key role in the inflammatory and immune responses. Levels of macrophage and monocyte-derived cytokines, including tumor necrosis factor alpha, interferon gamma, interleukin (IL)-1 alpha, IL-1 beta, IL-6, IL-10, as well as chemokines including Macrophage inflammatory protein-1 (MIP-1), and IL-8 were measured at the transcription level in vivo and normalized to a house keeping gene, glyceraldehyde-3-phosphate dehydrogenase (GAPDH).

THP-1 cells were grown in a supplier-recommended medium of RPMI 1640 medium with 2 mM L-glutamine adjusted to contain 1.5 g/L sodium bicarbonate, 4.5 g/L glucose, 10 mM HEPES and 1.0 mM sodium pyruvate and supplemented with 0.05 mM 2-mercaptoethanol, 90% fetal bovine serum, 10% at 37° C. (98.6° F.). The cells were seeded at a concentration of 10⁶ cells/mL 24 hours prior to experimentation. Cells were then treated with Echinacea extracts from plants harvested at maturation stages 1, 3 and 6. A lipopolysaccharide (LPS) solution was used as a positive control. The extracts were prepared in concentrations of 100 ug/ml (micrograms per milliliter) in growth media. The extracts were in a stock solution of 50% DMSO, 30% ethanol, 20% water to allow a final solvent concentration not exceeding 0.5% in treatment. LPS as a positive control was used at 500 ng/ml final concentration. Cell cultures were incubated at 37° C. (98.6° F.) for six hours prior to the cells being harvested.

The cells were harvested by centrifugation. The RNA was isolated using conventional Trizol/guanidine isothiocyanate based lysis buffer as instructed in the RNA isolation kit (#200345) from Stratagene (La Jolla, Calif.). The cell cultures may also be harvested by scraping or trypinizing methods.

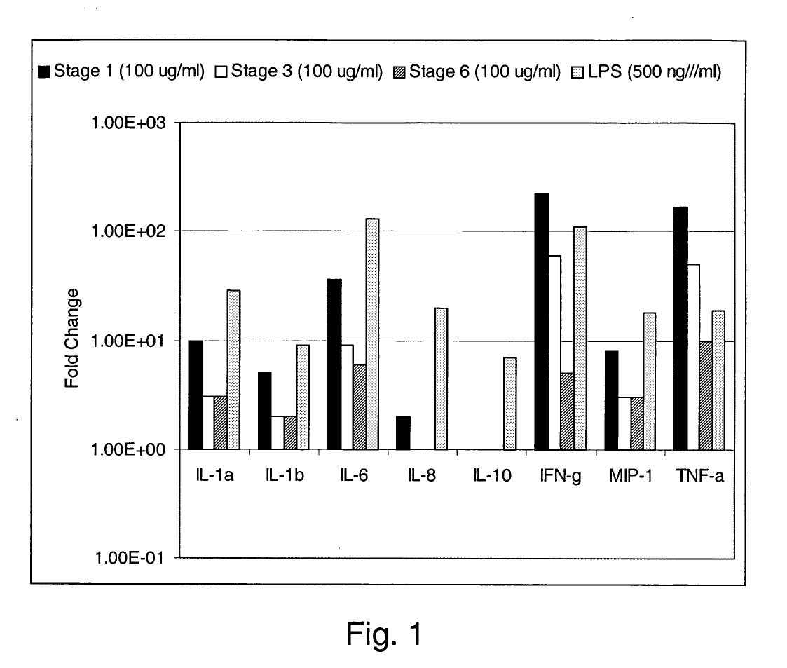

The induction of immune cytokine mRNA and chemokine mRNA were measured by well-known methods of quantitative reverse transcriptase-polymerase chain reaction (qRT-PCR). Baseline measurements were taken to be non-LPS stimulation and were compared to real time PCR data. The results are shown in FIG. 1. Fold induction is calculated by comparing the results to the level of expression of each gene in un-induced TIIP-1 cells.

When the three maturation stages of E. purpurea were compared for their immune stimulatory effects on THP-1 monocyte/macrophage cell line, stage 1 material exhibited the most potent induction activity, especially on induction of cytokines Interferon-gamma and Tumor necrosis factor alpha.

The chicoric acid levels of stages 1, 3 and 6 do not vary considerably, see Table 1. The results depicted in FIG. 1 indicate that while the levels of a standardization marker do not varying remarkably between maturation stages, immune-stimulatory induction vary a great deal based on the maturation stage during which the plant was harvested. Harvesting Echinacea plants prior to full bloom for use in products intended to stimulate the immune system does not interfere with currently practiced standardization procedures, but may provide for a greater immune-stimulatory effect.

Aside from the specific preparations discussed above, the standardization of marker levels and the augmentation of immune-stimulatory response are expected to be observed even if the preparation of plants varies provided the variation is not intended to remove phytochemicals from the preparation. For example, the augmented immune-stimulatory effects obtained from the consumption of phytochemicals derived from the harvest of Echinacea plants during maturation stages prior to full bloom are expected to also be observed for preparations of powdered Echinacea as described in U.S. Pat. No. 6,217,878 to Menon et al.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as” or “for example”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise.

1. A method for determining optimal harvest window of medicinal plants, the method comprising the steps of:
harvesting at least one plant at a plurality of maturation stages for the plant;
adding a preparation of the plant to a cell culture;
harvesting the cell culture;
analyzing the cell culture for a level of a product the medicinal plant induces; and
observing the level of product corresponding to each of the different maturation stages.

2. The method of claim 1, further comprising the step of determining a concentration of a marker compound for each of the plants at the plurality of maturation stages.

3. A method for determining optimal harvest window of Echinacea plants, the method comprising the steps of:
harvesting at least one plant at a plurality of maturation stages for the plant;
adding a preparation of the plant to a cell culture;
harvesting the cell culture;
analyzing the cell culture for a level of an immune-stimulatory product induced by Echinacea; and
observing the level of the immune-stimulatory product corresponding to each of the different maturation stages.

4. The method of claim 3, further comprising the step of determining a concentration of a marker compound for each of the plants at the plurality of maturation stages.

5. The method of claim 4 wherein the marker compound is selected from a group consisting of chicoric acid, alkylamides, glycoproteins, polysaccarides and combinations thereof.

6. The method of claim 3 wherein the immune-stimulatory product is selected from the group consisting of cytokine mRNA and chemokine mRNA.

7. The method of claim 3 wherein the immune-stimulatory product is an mRNA transcript selected from the group consisting of IL-1 alpha, IL-1 beta, IL-6, IL-8, IL-10, tumor necrosis factor alpha, interferon-gamma and macrophage inflammatory protein-1.

8. A method of augmenting the immune-stimulatory effects of Echinacea extracts, the method comprising the steps of:
harvesting an Echinacea plant during a maturation stage that includes stages prior to full bloom;
drying the plant;
reducing the plant size; and
extracting the plant with a solvent.

9. The method of claim 8 wherein the maturation stage is vegetative.

10. The method of claim 8 further comprising the step of maintaining a standardized level of chicoric acid.

11. A method of augmenting the immune-stimulatory effects of Echinacea extracts, the method comprising the steps of:
harvesting an Echinacea plant during a maturation stage that is vegetative drying the plant;
reducing the plant size; and
extracting the plant with a solvent.

12. An Echinacea preparation comprising:
a standardized concentration of chicoric acid; and
an augmented level of immune-stimulatory activity;
wherein the preparation was obtained from an Echinacea plant harvested during a maturation stage prior to full bloom.

13. The preparation of claim 12, wherein the augmented level of immune-stimulatory activity is measured by inducement in THP-1 cells of an mRNA transcript selected from the group consisting of: IL-1 alpha, IL-1 beta, IL-6, IL-8, IL-10, tumor necrosis factor alpha, interferon-gamma and macrophage inflammatory protein-1.

14. The preparation of claim 12, wherein the augmented level of immune-stimulatory activity is measured by inducement in THP-1 cells of an mRNA transcript selected from the group consisting of tumor necrosis factor alpha and interferon-gamma.

15. An Echinacea preparation comprising:
a standardized concentration of chicoric acid; and
an augmented level of immune-stimulatory activity;
wherein the preparation was obtained from a plant harvested during the vegetative stage.

16. The preparation of claim 15, wherein the augmented level of immune stimulatory activity is measured by inducement in THP-1 cells of an mRNA transcript selected from the group consisting of: IL-1 alpha, IL-1 beta, IL-6, IL-8, IL-10, tumor necrosis factor alpha, interferon-gamma and macrophage inflammatory protein-1.

17. The preparation of claim 15, wherein the augmented level of immune stimulatory activity is measured by inducement in THP-1 cells of an mRNA transcript selected from the group consisting of tumor necrosis factor alpha and interferon-gamma.

18. An Echinacea preparation comprising:
a standardized concentration of chicoric acid,
wherein the preparation induces an augmented level of immune-stimulatory activity; and
wherein the preparation was obtained from a plant harvested during a vegetative stage.

19. The preparation of claim 18, wherein the augmented level of immune-stimulatory activity is measured by inducement in THP-1 cells of an mRNA transcript selected from the group consisting of: IL-1 alpha, IL-1 beta, IL-6, IL-8, IL-10, tumor necrosis factor alpha, interferon-gamma and macrophage inflammatory protein- 1.

20. The preparation of claim 18, wherein the augmented level of immune-stimulatory activity is measured by inducement in THP-1 cells of an mRNA transcript selected from the group consisting of tumor necrosis factor alpha and interferon-gamma.

21. A preparation of Echinacea purpurea comprising:
a standardized level of chicoric acid of at least about 3.49 percent as measured by HPLC analysis;
wherein the preparation provides an augmented immune-stimulatory response in THP-1 cells of at least 100 times.

22. The preparation of claim 21 wherein the augmented immune-stimulatory response is measured by inducement in the cells of an mRNA transcript selected from the group consisting of tumor necrosis factor-alpha and interferon-gamma.
